(19) United States
(12) Patent Application Publication
(10) Pub. No.: US 2024/0052396 A1
(43) Pub. Date: Feb. 15, 2024

(54) SELECTIVE PROTECTION OF NUCLEIC ACIDS

(71) Applicant: Harbinger Health, Inc., Cambridge, MA (US)

(72) Inventor: Anthony P. Shuber, Northbridge, MA (US)

(21) Appl. No.: 18/124,212

(22) Filed: Mar. 21, 2023

Related U.S. Application Data
(63) Continuation of application No. 16/007,510, filed on Jun. 13, 2018, now abandoned.
(60) Provisional application No. 62/656,599, filed on Apr. 12, 2018.
(60) Provisional application No. 62/526,091, filed on Jun. 28, 2017.
(60) Provisional application No. 62/519,051, filed on Jun. 13, 2017.

(51) Int. Cl. C12Q 1/6806; C12N 9/22; C12N 15/11

(52) U.S. Cl. CPC C12Q 1/6806; C12N 9/22; C12N 15/11; C12N 2800/80; C12N 2310/20

(57) ABSTRACT

The invention provides methods of selectively protecting nucleic acids of interest in a sample from damage that occurs during preparative procedures. The methods include binding proteins to ends and to one or more internal regions of a segment of the nucleic acid of interest so that damage to exposed regions of the segment does not lead to degradation of the entire segment.

### CROSS-REFERENCE TO RELATED APPLICATIONS

This application claims the benefit of, and priority to, US. Application No. 62/656,599 filed on Apr. 12, 2018, U.S. Application No. 62/526,091 filed on Jun. 28, 2017, and U.S. Application No. 62/519,051 filed on Jun. 13, 2017, the contents of each of which are incorporated by reference.

### FIELD OF THE INVENTION

The disclosure relates to molecular genetics.

### BACKGROUND

DNA has the potential to provide significant clinical diagnostics information. For example, DNA obtained from a tumor can reveal whether a cancer patient is in remission, or may inform a physician about immunotherapy treatments that may be effective for the patient. It is also possible to detect cell-free circulating DNA in blood as a means for obtaining minimally-invasive diagnostic information. Similarly, fetal DNA can be obtained from a mothers plasma to assess certain genetic disorders, aneuploidy, or the risk of preeclampsia.

Existing methods for obtaining nucleic acid in the context of liquid biopsy can be problematic due to the inability to obtain an amount of circulating nucleic acid sufficient for diagnostic purposes. In addition, there is a significant signal-to-noise problem that makes the separation of the small amount of diagnostically-relevant nucleic acid from the significant amount of background difficult.

### SUMMARY

The invention provides methods of tiling proteins along a target nucleic acid in a sample to protect the target while isolating the target from the sample, to increase the probability of obtaining and analyzing diagnostically-relevant sequence. Methods of the invention use binding proteins such as Cas endonuclease or catalytically inactive Cas endonuclease to bind to target nucleic acid and inhibit degradation of the target nucleic acid. In a preferred example, protein complexes are bound to the ends of a nucleic acid segment interest and additional binding proteins are interspersed in the intervening sequence to inhibit degradation of target sequence by digestion, nicking and other enzymes present in the biological sample from which the nucleic acid is obtained.

Methods of the invention are useful in a wide variety of applications. For example, because methods of the invention preserve target sequence, they are ideal for detection of sequence that is present in a sample at low abundance. Thus, methods of the invention are useful for analysis of cfDNA in blood or blood products (e.g., plasma). As a result, methods of the invention allow the early detection of genomic alterations indicative of cancer and identification of genetic disorders of a fetus in utero. Methods of the invention are also ideal for the analysis of large DNA fragments that are typically degraded in sample preparation processes of the art. For example, nucleic acids are likely to sustain nicks during sample preparation, which can lead to degradation. Methods of the invention confine damage resulting from degradation during preparation to localized portions of a target segment so that useful information can be obtained from the remaining portions. Consequently, methods of the invention allow detection of genomic alterations, big and small, such as duplications, translocations, LOH, inversions and the like. In one aspect, the invention provides methods of protecting a target nucleic acid in or prepared from a biological sample by binding proteins to ends of the target and binding one or more additional proteins along the interstitial length of the target.

Each of the end-binding proteins may independently be any protein that hinds a nucleic acid in a sequence-specific manner. In a preferred embodiment, end-binding proteins are programmable nucleases. For example, end-binding proteins may be a CRISPR-associated (Cas) endonuclease, zinc-finger nuclease (ZFN), transcription activator-like effector nuclease (TALEN), or RNA-guided engineered nuclease (ROEN). These proteins may be a catalytically inactive form of a nuclease, such as a programmable nuclease described above. In addition, proteins such as a transcription activator-like effector (TALE) may be used.

A preferred protein for end binding is a Cas endonuclease, Cas is complexed with target nucleic acid using guide RNAs that are designed for sequence-specific binding. Proteins for binding between the ends may also be Cas proteins or others that protect sequence from degradation. An ideal protein is catalytically-inactive (dead) Cas (dCas). Whatever binding protein modality is used, the intermediate regions (i.e., between the ends) must be tiled along the sequence at spaced intervals designed to decrease degradation of the target sequence.

Methods of the invention further include detecting the target sequence. Binding proteins may be removed prior to detection. The undamaged portion (i.e., that portion that was protected or otherwise not degraded during sample acquisition or sample preparation) of a target may be detected by any means known in the art. For example and without limitation, the intact portion may be detected by DNA staining, spectrophotometry, sequencing, fluorescent probe hybridization, fluorescence resonance energy transfer, optical microscopy, or electron microscopy.

Detection methods may include mapping or comparing detected sequence to a reference. Sequence read length depends upon the integrity of the sample obtained. A sequence can be compiled using known bioinformatic methods.

Nucleic acid for analysis may be obtained from any sample type, such as a liquid or body fluid from a subject, such as urine, blood, plasma, serum, sweat, saliva, semen, feces, phlegm, or a liquid biopsy. The sample may be a food sample. The sample may be from an environmental source, such as a soil sample, or water sample.

The nucleic acid of interest may contain a mutation. For example and without limitation, the feature may be an insertion, deletion, substitution, inversion, amplification, duplication, translocation, or polymorphism. The nucleic acid of interest may be from an infectious agent or pathogen. For example, the nucleic acid sample may be obtained from an organism, and the nucleic acid of interest may contain a sequence foreign to the genome of that organism. The nucleic acid of interest may be from a sub-population of nucleic acid within the nucleic acid sample. For example, the nucleic acid of interest may be cell-free DNA, such as cell-free fetal DNA or circulating tumor DNA.

The nucleic acid may be any naturally-occurring or artificial nucleic acid. The nucleic acid may be DNA, RNA, hybrid DNA/RNA, peptide nucleic acid (PNA), morpholino and locked nucleic acid (LNA), glycol nucleic acid ((INA), threose nucleic acid (TNA), or Reno nucleic acid. The RNA may be a subpopulation of RNA, such as mRNA, tRNA, rRNA, miRNA, or siRNA. Preferably the nucleic acid is DNA.

The method may include digesting the nucleic acid. The nucleic acid may be digested with a nuclease.

Embodiments of the disclosure involve the isolation or extraction of a nucleic acid from a tissue sample such as, for example, a formalin-fixed, paraffin embedded (FFPE) tissue sample. Such embodiments provide methods that include selectively protecting target nucleic acid in a tissue sample (e.g., an FFPE tissue sample), and extracting the nucleic acid from the tissue sample, to obtain or analyze diagnostically-relevant sequence. Extracting nucleic acid from tissue samples may include binding protein complexes to the ends of a nucleic acid of interest and optionally binding additional binding proteins to the intervening sequence, before or while performing enzymatic proteolysis under optimized conditions, followed by solid-phase extraction of the nucleic acid on glass-fiber or other solid supports. Methods may include binding the proteins along the target nucleic acid, e.g., introducing proteins that will “tile” along a target nucleic acid. Cas endonuclease may be used and it may be preferable to use catalytically inactive Cas endonuclease (“dCas”). The dCas proteins can be introduced along with guide RNAs that target the dCas to intended positions along the nucleic acid. For example, it may be preferable to target dCas to both ends of the target nucleic acid and optionally also to tile the dCas proteins along the nucleic acid, i.e., to bind the dCas proteins at a series of intervening positions between the two ends. So protecting the nucleic acid may be done in conjunction with isolating or extracting the nucleic acid from the tissue sample using any suitable method, such as using an FFPE nucleic acid extraction kit or reagents from a commercial provider. The methods include removing paraffin wax and using a solid phase for final extraction of the nucleic acid. Replacement of the wax may be done using water through a series of soaks in xylene (or limonene) and various dilutions of ethanol in water. Paraffin wax removal may be done by the direct incubation of a slice of the embedded tissue in proteolytic solution. Final extraction may use a glass-fiber filter in a spin-column format for the solid phase recovery step, magnetic beads, or any other suitable solid phase extraction.

### BRIEF DESCRIPTION OF THE DRAWINGS

FIG. 1 is a diagram of a method according to an embodiment of the invention.

FIG. 2 diagrams a method of detecting a nucleic acid.

FIG. 3 shows the detection of a nucleic acid of interest.

### DETAILED DESCRIPTION

The invention provides methods of selectively protecting a nucleic acid segment within a sample by binding proteins to the ends of the segment and to one or more internal regions. Damage may occur to portions of the segment that are not bound to proteins, but binding of the proteins prevents damage, e.g., degradation, from spreading along the length of the segment. Consequently, portions of the segment are protected from degradation. Therefore, undegraded portions can be detected and analyzed.

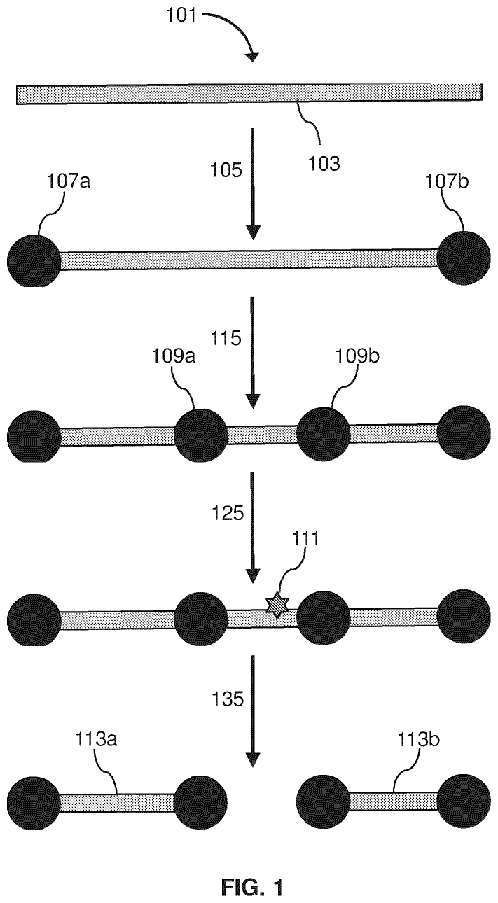

FIG. 1 is a diagram of a method 101 according to an embodiment of the invention. A segment 103 of a nucleic acid of interest is provided within a nucleic acid sample. Proteins 107a and 107b are bound 105 to the ends of the segment 103. Additional proteins 109a and 109b are bound 115 along the length of the segment 103. Preferably, the proteins 107a, 109a, 109b, and 107b, and others are “tiled” along the nucleic acid. Any number of proteins 109a and 109b be tiled along the segment 103. Preferably, the proteins 109a and 109b are tiled with regular or approximately regular spacing. The number and spacing of proteins 109a and 109b may vary depending on the length of segment 103. Binding steps 105 and 115 may be performed simultaneously or sequentially in either order. An exposed portion of the segment 103, i.e., a portion that is not bound to a protein, is damaged 125, creating a nick 111. As a result of the nick, the damaged portion of the segment is degraded 135. However, due to the binding of proteins 109a and 109b degradation 135 of the segment is halted at the sites where proteins 109a and 109b are bound. Consequently, damage is confined to a localized area, and portions 113a and 113b remain intact. Therefore, the intact portions can be detected to provide useful information about the segment 103.

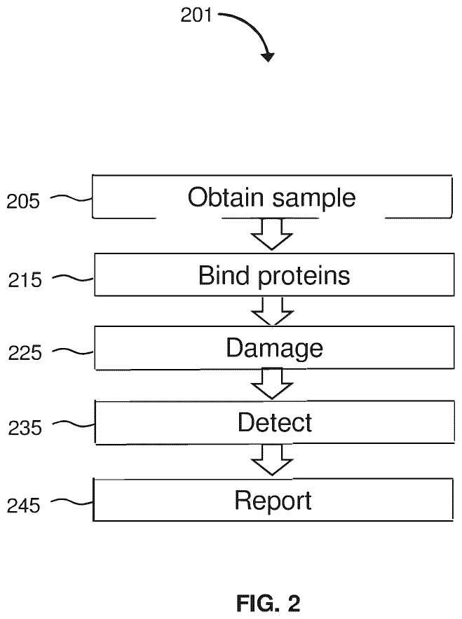

FIG. 2 diagrams a method 201 of detecting a nucleic acid according to a method of the invention. The method 201 may include obtaining 205 a nucleic acid sample. Proteins and then bound 215 to a segment of a nucleic acid of interest as described above. The nucleic acids in the sample, including one or more unbound portions of the segment, are then damaged 225. Damage may occur incidentally as an artifact of sample preparation, or it may be actively initiated. Damage leads to degradation of stretches that do not have bound proteins, but undamaged regions that are flanked by bound proteins are protected from degradation. Intact portions of the segment may then be detected 235. The method 201 may include reporting 245 that one or more portions of the segment are present in the sample.

The nucleic acid may be any naturally-occurring or artificial nucleic acid. The nucleic acid may be DNA, RNA, hybrid DNA/RNA, peptide nucleic acid (PNA), morpholino and locked nucleic acid (INA), glycol nucleic acid (GNA), threose nucleic acid (TNA), or Xeno nucleic acid. The RNA may be a subpopulation of RNA, such as snRNA, tRNA, rRNA, miRNA, or siRNA. Preferably the nucleic acid is DNA.

The damage may be any type of damage that occurs during processing of nucleic acids. A common type of DNA damage is the breakage of the phosphodiester bonds between adjacent nucleotides in the same strand, called a nick. Other types of damage to DNA include double-strand breaks, base excision, and nucleotide excision. As indicated above, the damage may occur adventitiously during processing of nucleic acids. Alternatively or additionally, it may occur through active, deliberate manipulation of the sample, for example by treating the sample with a nickase. Additional enzymes, such as nucleases, e.g., exonucleases and endonucleases, may be added to facilitate degradation.

The choice of whether to rely on passive or deliberate nicking depends on the particular application of the method. For example, to analyze certain genetic events, such as translocations and duplications, large segments of DNA must be analyzed. However, larger DNA fragments or more susceptible to nicking. Therefore, it is desirable to minimize damage when analyzing large fragments (e.g., >50 kb), so deliberate nicking should be avoided. On the other hand, when the nucleic acid of interest is present in small quantities, i.e., at low abundance, in the sample, it may be desirable to actively induce nicking or other types of damage to remove irrelevant species and thus enrich the sample for the nucleic acid of interest. This method has been called negative enrichment and is described in co-pending, co-owned U.S. application Ser. Nos. 15/877,619 and 15/877,620, the contents of which are incorporated herein by reference.

Damage to unprotected nucleic acids may result in a population of partially degraded nucleic acid molecules of a certain size. For example, the damaged nucleic acids may be less than about 10 nucleotides, less than about 20 nucleotides, less than about 50 nucleotides, less than about 100 nucleotides, less than about 200 nucleotides, less than about 500 nucleotides, less than about 1000 nucleotides, less than about 2000 nucleotides, or less than about 5000 nucleotides. Digested nucleic acids may be smaller than the nucleic acid of interest. All or substantially all unprotected nucleic acids may be degraded. For example, at least 90%, at least 95%, at least 98%, at least 99%, at least 99.9% of unprotected nucleic acids may be degraded.

The proteins that bind to the segment may be any proteins that bind a nucleic acid in a sequence-specific manner, Some or all of the proteins may be the same, or each protein may be different. Preferably, the proteins that bind to the ends of segment are the same. In some embodiments, all of the proteins, i.e., the proteins that bind the ends and the proteins that bind internal regions, are the same.

The protein may be a programmable nuclease. For example, the protein may be a CRISPR-associated (Cas) endonuclease, zinc-finger nuclease (ZFN), transcription activator-like effector nuclease (TALEN), or RNA-guided engineered nuclease (RGEN). Programmable nucleases and their uses are described in, for example, Zhang F, Wen Y, Guo X (2014). “CRISPR/Cas9 for genome editing: progress, implications and challenges”. Human Molecular Genetics. 23 (R1): R40-6. doi:10.1093/hmg/ddu125; Ledford H (March 2016). “CRISPR: gene editing is just the beginning”. Nature. 531 (7593): 156-9. doi:10.1038/5311.56a; Hsu P D, Lander E S, Zhang F (June 2014). “Development and applications of CRISPR-Cas9 for genome engineering”. Cell. 157 (6): 1262-78. doi:10.1.016/j.cell.2014.05.010; loch J (February 2011). “TALEs of genome targeting”. Nature Biotechnology. 29 (2): 135-6. doi:10.1038/nbt.1767; Wood A J, Lo T W, Zeitler B, Pickle C S, Ralston E J, Lee A H, Amora R, Miller J C, Leung E, Meng X, Zhang L, Rebar E J, Gregory P D, Urnov F D, Meyer B J (July 2011). “Targeted genome editing across species using ZFNs and TALENs”. Science. 333 (6040): 307. doi:10.1126/science.1.207773; Carroll, D (2011). “Genome engineering with zinc-finger nucleases”, Genetics Society of America, 188 (4): 773-782. doi:10.1534/genetics.111.131433; Urnov, F. D., Rebar, J., Holmes, M. C., Zhang, H. S., Gregory, P. D. (2010). “Genome Editing with Engineered Zinc Finger Nucleases”. Nature Reviews Genetics. 11 (9): 636-646. doi:10.1038/nrg2842, the contents of each of which are incorporated herein by reference. In a preferred embodiment, the binding proteins 107 and 109 are provided by Cas endonucleases. Any suitable Cas endonuclease or homolog thereof may be used. A Cas endonuclease may be Cas9 (e.g., spCas9), catalytically inactive Cas (dCas such as dCas9), Cpf1, C2c2, others, modified variants thereof, and similar proteins or macromolecular complexes. The protein may be a catalytically inactive form of a nuclease, such as a programmable nuclease described above. The protein may be a transcription activator-like effector (TALE).

The protein may be complexed with a nucleic acid that guides the protein to an end of the segment. For example, the protein may be a Cas endonuclease in a complex with a guide RNA. In an embodiment, proteins 107a and 107b are complexed with guide RNAs that have sequences complementary to the ends of the segment, and proteins 109a and 109b are complexed with guide RNAs that have sequences complementary to regions in the interior of the segment. For example and without limitation, the Cas endonuclease may be Cas9, Cpf1, C2c1, C2c3, C2c2, CasX, or CasY, including sequence variants of Cas9, Cpf1, C2c1, C2c3, C2c2, CasX, or CasY. Preferably, the Cas endonuclease is Cas9, The Cas endonuclease may be from any bacterial species. For example and without limitation, the Cas endonuclease may be from Bacteroides coprophilus, Campylobacter jejuni susp. jejuni, Campylobacter lari, Fancisella novicida, Filifactor aiocis, Flavobacterium columnare, Fluviicola taffensis, Eiluconacetobacter diazotrophicus, Lactobacillus farciminis, Lactobacillus johnsonii (e), Legionella pneumophila, Mycoplasma gallisepticum, Mycoplasma mobile, Neisseria cinerea. Neisseria meningitidis, Nitratifractor salsuginis, Parvibaculum lavamentivorans, Pasteurella multocida, Sphaerochaeta globusa, Streptococcus pasteurianus, Streptococcus thermophilus, Sutterella wadsworthensis, and Treponema denticola.

A guide RNA mediates binding of the Cas complex to the guide RNA target site via a sequence complementary to a sequence in the target site. Typically, guide RNAs that exist as single RNA species comprise a CRISPR (cr) domain that is complementary to a target nucleic acid and a tracr domain that, binds a CRISPR/Cas protein. However, guide RNAs may contain these domains on separate RNA molecules.

The ends of the segment, which are bound by proteins, may be separated by any distance. For example, the ends may be within at least at least 500 bases, at least 1000 bases, at least 2000 bases, at least 5000 bases, at least 10,000 bases, at least 20.000 bases, at least 50,000 bases, at least 100,000 bases, at least 200,000 bases, at least 500,000 bases, at least one megabase, or at least two megabases of each other.

The nucleic acid sample may come from any source. For example, the source may be an organism, such as a human, non-human animal, plant, or other type of organism. The sample may be a tissue sample from an animal, such as blood, serum, plasma, skin, urine, saliva, semen, feces, phlegm, conjunctiva, gastrointestinal tract, respiratory tract, vagina, placenta, uterus, oral cavity or nasal cavity. The sample may be a liquid biopsy.

The nucleic acid sample may come from an environmental source, such as a soil sample or water sample, or a food source, such as a food sample or beverage sample. The sample may comprise nucleic acids that have been isolated, purified, or partially purified from a source. Techniques for preparing nucleic acids from tissue samples and other sources are known in the art and described, for example, in Green and Sambrook, Molecular Cloning: A Laboratory Manual (Fourth Edition), Cold Spring Harbor Laboratory Press, Woodbury, NY 2,028 pages (2012), incorporated herein by reference. Alternatively, the sample may not have been processed.

The sample may contain cell-free DNA, such as circulating tumor DNA (ctDNA) or fetal DNA from maternal blood or plasma. Circulating tumor DNA exists in the blood of some patients in early stages of cancer, so detection and analysis of circulating tumor DNA is useful for diagnosis of cancer. In some embodiments, the sample includes at least one circulating tumor cell from a tumor and the segment comprises tumor DNA from the tumor cell. Cell-free fetal DNA (cffDNA) exists in the blood of pregnant females, so detection and analysis cffDNA is useful for diagnosing genetic defects while the fetus is in utero.

Methods may include detection or isolation of circulating tumour cells (CTCs) from a blood sample. Cytometric approaches use immunostaining profiles to identify CTCs. CTC methods may employ an enrichment step to optimize the probability of rare cell detection, achievable through immune-magnetic separation, centrifugation or filtration. Cytometric CTC technology includes the CTC analysis platform sold under the trademark CELLSEARCH by Veridex LLC (Huntingdon Valley, PA). Such systems provide semi-automation and proven reproducibility, reliability, sensitivity, linearity and accuracy. See Krebs, 2010, Circulating tumor cells, Ther Adv Med Oncol 2(6):351-365 and Miller, 2010, Significance of circulating tumor cells detected by the CellSearch system in patients with metastatic breast colorectal and prostate cancer, J Oncol 2010:617421-617421, both incorporated by reference.

The segment may be detected by any means known in the art. For example and without limitation, the segment may be detected by DNA staining, spectrophotometry, sequencing, fluorescent probe hybridization, fluorescence resonance energy transfer, optical microscopy, or electron microscopy. Methods of DNA sequencing are known in the art and described in, for example, Pettersson E, Lundeberg J, Ahmadian A (February 2009), “Generations of sequencing technologies”. Genomics. 93 (2): 105-11. doi:10.1016/j.ygeno.2008.10.003; Goodwin, Sara; McPherson, John D.; McCombie, W. Richard (17 May 2016). “Coming of age: ten years of next-generation sequencing technologies”. Nature Reviews Genetics. 17 (6): 333-51. doi:10.1038/nrg.2016.49; and Morey M, Fermández-Marmiesse A, Castiñeiras D, Fraga J M, Couce M L, Cocho J A (2013). “A glimpse into past, present, and future DNA sequencing”. Molecular Genetics and Metabolism. 110 (1-2): 3-24. doi:10.1016/j.ymgme.201.3.04.024. Other methods of DNA detection are known in the art and described in, for example, Xu et al., Label-Free DNA Sequence Detection through FRET from a Fluorescent Polymer with Pyrene Excimer to SG, ACS Macro Lett., 2014, 3 (9), pp 845-848, DOI: 1.0.1021/mz500378c; and Green and Sambrook, eds., Molecular Cloning: A Laboratory Manual, 4th edition, Cold Spring Harbor Press, Cold Spring Harbor, N Y, 2012, ISBN 978-1-936113-41-5.

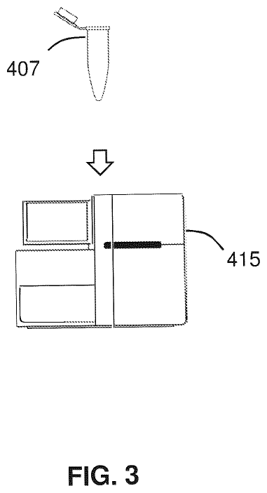

FIG. 3 shows the detection of a nucleic acid of interest. The nucleic acid may be provided as an aliquot 407 (e.g., in a micro centrifuge tube such as that, sold under the trademark EPPENDORF by Eppendorf North America (Hauppauge, NY) or glass cuvette). The nucleic acid may be disposed on a substrate. For example, the nucleic acid may be pipetted onto a glass slide and subsequently combed or dried to extend it across the glass slide. The nucleic acid may optionally be amplified. Optionally, adaptors are ligated to ends of the nucleic acid, which adaptors may contain primer sites or sequencing adaptors. The presence of the nucleic acid may then be detected using an instrument 415.

The nucleic acid may be detected, sequenced, or counted. When multiple nucleic acids of interest are present, they may be quantified, e.g., by qPCR.

In certain embodiments, the instrument 415 is a spectrophotometer, and detection includes measuring the adsorption of light by the nucleic acid. The method may be performed in fluid partitions, such as in droplets on a microfluidic device, such that each detection step is binary (or “digital”). For example, droplets may pass a light source and photodetector on a microfluidic chip and light may be used to detect the presence of a segment of DNA in each droplet (which segment may or may not be amplified as suited to the particular application circumstance). By the described methods, a sample can be assayed using a technique that is inexpensive, quick, and reliable. Methods of the disclosure are conducive to high throughput embodiments, and may be performed, for example, in droplets on a microfluidic device, to rapidly assay a large number of aliquots from a sample for one or any number of genomic structural alterations.

The methods may include providing a report to a subject or patient. The report preferably includes information about the subject's condition, such as a diagnosis, prognosis, or suggested course of therapy. Knowledge of a mutational landscape of a tumor may be used to inform treatment decisions, monitor therapy, detect remissions, or combinations thereof. For example, where the report includes a description of a plurality of mutations, the report may also include an estimate of a tumor mutation burden (TMB) for a tumor. It may be found that TMB is predictive of success of immunotherapy in treating a tumor, and thus methods described herein may be used for treating a tumor.

Analysis may include sequencing multiple portions of a segment and creating a map of a larger region of the segment. By aligning overlapping sequences from adjacent portions, an extended continuous sequence of the segment can be obtained from small portions. Thus, useful information can be obtained from the segment even if isolation of large, intact portions of the segment is not possible. Mapping may be used to reconstruct a region of the segment that is at least 100 bases, at least 200 bases, at least 500 bases, at least 1000 bases, at least 2000 bases, at least 5000 bases, at least 10,000 bases, at least 20,000 bases, at least 50,000 bases, or at least 100,000 bases in length.

The nucleic acid of interest may contain information that provides insight about a disease or condition. For example and without limitation, the nucleic acid of interest may contain an insertion, deletion, substitution, inversion, amplification, duplication, translocation, copy number variation, or polymorphism. The nucleic acid of interest may be from an infectious agent or pathogen. For example, the nucleic acid sample may be obtained from an organism, and the nucleic acid of interest may contain a sequence foreign to the genome of that organism.

The nucleic acid of interest may be from the genome of a pathogen, such as a virus, bacterium, or fungus. The population of nucleic acids may come from an organism, and the nucleic acid of interest may be foreign to the genome of the organism. For example, the nucleic acid of interest may be from a pathogen of the organism. The nucleic acid of interest may be from a virus that infects the organism from which the nucleic acids are obtained. The nucleic acid of interest may be a viral nucleic acid that has integrated into the genome of the host organism. Additionally or alternatively, the nucleic acid of interest may be a viral nucleic acid that exists separately from the nucleic acids of the host organism. The nucleic acid of interest may be native to the organism from which the population has been obtained. For example, the nucleic acid of interest may be from the nuclear genome, mitochondrial genome, or chloroplast genome of the organism. The population of nucleic acids may come from a tissue sample from an organism, and the nucleic acid of interest may be a nucleic acid that is present in that tissue in a low abundance and/or is indicative of a pathological or medical condition.

The methods are also useful for detecting nucleic acid from an infectious agent, such as a virus, that may be present in a host, Methods are addressed to challenges by which viral nucleic acid may be difficult to detect among abundant host DNA. Thus the detected nucleic acid may be from the genome of an infecting virus. Obtaining the sample may include taking a tissue sample from a patient and extracting or accessing DNA therein. The DNA of an infecting virus is isolated by digesting away substantial amounts of non-viral DNA. For example, viral DNA may be protected, while host DNA is damaged and degraded, and detection of intact, portions of viral DNA indicates the presence of the virus in the host. The detected viral DNA may be of any suitable virus including retroviruses that integrate into the host genome and virus present as viral episomes.

In a preferred embodiment, the method includes providing guide RNAs that are specific to a viral genome, such as HIV and using those guide RNAs with Cas endonuclease to protect a segment of viral DNA in a sample from a patient. After digesting away unprotected. DNA, the remaining DNA is detected, confirming the presence of the virus in the host genome. The methods thus provide a rapid and reliable viral test, that can detect retroviral proviral DNA and/or viral episomes, and thus detect viral infections at any stage.

In certain embodiments, the methods have applications in metagenomics. Metagenomics includes to the study of genetic material recovered directly from environmental samples. While traditional microbiology and microbial genome sequencing and genomics relied upon cultivated clonal cultures, early environmental gene sequencing cloned specific genes (often the 16S rRNA gene) to produce a profile of diversity in a natural sample. Here, methods of the invention are useful to take essentially unbiased samples of all genes from all the members of the sampled communities. Because of their ability to reveal the previously hidden diversity of microscopic life, methods of the disclosure applied to metagenomics offers novel views of the microbial world. Specifically, using a mixture of guide RNAs, the method may be used to isolate a plurality of representative sample fragments of microbial DNA from an environmental sample, such that the set of sample fragments are a representative sample of microbial diversity in the environmental sample. In some embodiments, the methods are performed with an abundance of pseudo-random guide RNAs in Cas endonuclease complexes. Pairs of the complexes isolate DNA fragments from an environmental sample. The collected set of fragments is essentially a vertical slice through the microbial genetic information in the sample, and thus is representative of microbial diversity therein. Those fragments may be analyzed to reveal microbial diversity in the sample, even without culturing individual microbes or knowing a priori the species that may be present in the sample. The fragments may be analyzed by, for example, sequencing. Since methods of the invention are useful to isolate long, intact nucleic acid molecules, the methods are particularly useful for long-read, single-molecule sequencing platforms such as those of Oxford Nanopore or Pacific Biosciences. Using such a sequencing platform with a method of the invention, one may perform a metagenomic survey of a sample and microbial ecology may thus be investigated at a much greater scale and detail than before.

Methods of the invention can be used to identify the microbiome, i.e., the collective genetic material of the microbiota. The microbiota is the ecological community of commensal, symbiotic and pathogenic microorganism found in and on all multicellular organisms studied to date. A microbiota includes bacteria, archaea, protists, fungi and viruses. Microbiota have been found to be crucial for immunologic, hormonal, and metabolic homeostasis of their host. The symbiotic relationship between a host and its microbiota shapes the immune system of mammalians, insects, plants, and aquatic organisms.

Embodiments of the invention include identification of a microbiome from a sample. Such embodiments include a method of detecting a nucleic acid of interest. The methods may include detecting multiple microbes in a sample, Preferably, methods include protecting one or both ends of a nucleic acid from a genome of a microbe in a sample using a first Cas9 complex and a second Cas9 complex, degrading unprotected nucleic acids, and detecting at least one protected nucleic acid, thereby detecting the microbe in the sample. Multiple nucleic acids from multiple microbes can be detected using multiple of Cas9 complexes, allowing the microbiome of a sample to be determined. The method may include reporting the microbiome of a sample.

Embodiments of the disclosure involve the isolation or extraction of a nucleic acid from a tissue sample such as, for example, a formalin-fixed, paraffin embedded (FFPE) tissue sample. Such embodiments provide methods that include selectively protecting target nucleic acid in a tissue sample (e.g., an FFPE tissue sample), and extracting the nucleic acid from the tissue sample, to obtain or analyze diagnostically-relevant sequence. Methods of the invention utilize binding proteins that selectively bind to target sequence and prevent or inhibit degradation of target nucleic acid. In a preferred example, protein complexes are bound to the ends of a nucleic acid segment of interest and additional binding proteins are interspersed in the intervening sequence to inhibit degradation of target sequence by physical, chemical, or enzymatic damage, such as by enzymes present in the tissue sample from which the nucleic acid is obtained.

The collection of tissue samples for subsequent microscopic examination usually entails the use of formaldehyde as a fixative. In tissue, the initial reaction of the formaldehyde is thought to occur primarily with lysine residues of proteins, forming groups that react with Tyr, Trp, and His. Such reactions provide a stable specimen that maintains the tissue's structure and withstands processing and sectioning, it creates a problem for retrieving any of these “fixed” molecules. Although some reversal of the crosslinks by chemical methods is possible, the final products of the fixation reactions normally possess a stability that inhibit retrieval of nucleic acids, for background, see Masuda, 1999, Analysis of chemical modification of RNA from formalin-fixed samples and optimization of molecular biology applications for such samples, Nucleic Acids Res 27:4436-4443, incorporated by reference.

Extracting nucleic acid from tissue samples may include binding protein complexes to the ends of a nucleic acid of interest and optionally binding additional binding proteins to the intervening sequence, before or while performing enzymatic proteolysis under optimized conditions, followed by solid-phase extraction of the nucleic acid on glass-fiber or other solid supports. Methods may include binding the proteins along the target nucleic acid, e.g., introducing proteins that will “tile” along a target nucleic acid. Cas endonuclease may be used and it may be preferable to use catalytically inactive Cas endonuclease (“dCas”). The dCas proteins can be introduced along with guide RNAs that target the dCas to intended positions along the nucleic acid. For example, it may be preferable to target dCas to both ends of the target nucleic acid and optionally also to tile the dCas proteins along the nucleic acid, i.e., to bind the dCas proteins at a series of intervening positions between the two ends. So protecting the nucleic acid may be done in conjunction with isolating or extracting the nucleic acid from the tissue sample using any suitable method, such as using an FFPE nucleic acid extraction kit or reagents from a commercial provider.

Kits are commercially available for the preparation of both RNA and DNA from FFPE tissue include, for example, the nucleic acid isolation kit sold under the trademark RECOVERALL (Cat. No. AM1975) or the nucleic acid isolation kit sold under the trademark MAGMAX (Cat. No. 4463365), both sold by ThermoFisher Scientific (Waltham, MA). When used according to the disclosure, those kits provide methods that include: binding proteins (e.g., dCas complexed with guide RNA) along the target nucleic acid; performing proteolytic digestions under conditions (e.g., T) specified by protocols of the kit, followed by purification with solid-phase extraction.

The methods include removing paraffin wax and using a solid phase for final extraction of the nucleic acid. Replacement of the wax may be done using water through a series of soaks in xylene (or limonene) and various dilutions of ethanol in water. Paraffin wax removal may be done by the direct incubation of a slice of the embedded tissue in proteolytic solution. Final extraction may use a glass-fiber filter in a spin-column format for the solid phase recovery step, magnetic beads, or any other suitable solid phase extraction.

Other approaches to tiling proteins along a nucleic acid and extracting the nucleic acid from a tissue sample are within the scope of the disclosure.

The extraction process may begin with the removal of paraffin before, during, or after introducing the proteins that tile along the target. Paraffin removal may be performed in a passive manner using xylene or another type of solvent to dissolve the paraffin. There is also a so-called active technique that utilizes acoustic energy to de-paraffin tissue specimens.

After tissues are digested and release the protein-bound nucleic acid (e.g., the target with dCas bound along it), the target is bound to magnetic beads. The beads are separated and the captured products washed with an ethanol solution, then isopropanol to remove contaminants, then once more with ethanol, followed by nucleic acid elution from the magnetic particles.

Target nucleic acid bound with protein such as dCas can be extracted using column extraction using, e.g., materials and reagents from QIAGEN or Roche. For column extraction, nucleic acids are released from tissue sections using special lysis conditions before or while being bound to proteins such as dCas, then applied to a glass fiber column which immobilizes the target while contaminants are removed.

Target nucleic acid bound with protein such as dCas can be extracted using lysis only using materials from Epicentre Biotechnologies. The lysis method uses salt precipitation for extracting nucleic acids. Other extraction methods include plate-based solid-phase reversible binding technology (Azco). Using solid surface reversible binding plate technology, protein-bound nucleic acid is bound to plate walls and so isolated from other proteins, salts, and debris for easy purification.

The invention provides kits for performing the methods of the invention. The kit may include reagents for performing the step described herein. The reagents may include one or more binding proteins, such as Cas endonucleases, and guide RNAs. The kit may include one or more enzymes to promote damage, such as a nickase or nuclease. The kit may also include instructions 919 or other materials such as pre-formatted report shells that receive information from the methods to provide a report (e.g., by uploading from a computer in a clinical services lab to a server to be accessed by a geneticist in a clinic to use in patient counseling). The reagents, instructions, and any other useful materials may be packaged in a suitable container. Kits of the invention may be made to order. For example, an investigator may use, e.g., an online tool to design guide RNA and reagents for the performance of methods. The guide RNAs may be synthesized using a suitable synthesis instrument. The synthesis instrument may be used to synthesize oligonucleotides such as gRNAs or single-guide RNAs (sgRNAs). Any suitable instrument or chemistry may be used to synthesize a gRNA, In some embodiments, the synthesis instrument is the MerMade 4 DNA/RNA synthesizer from Bioautomation (Irving, TX). Such an instrument can synthesize up to 12 different oligonucleotides simultaneously using 50, 200, or 1,000 nanomole prepacked columns. The synthesis instrument can prepare a large number of guide RNAs per run. These molecules (e.g., oligos) can be made using individual prepacked columns (e.g., arrayed in groups of 96) or well-plates. The resultant reagents (e.g., guide RNA, endonuclease(s), exonucleases) can be packaged in a container for shipping as a kit.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of protecting a nucleic acid, the method comprising:
binding a first protein and a second protein to ends of a segment of nucleic acid of interest;
binding one or more additional proteins along a length of the segment between the first and second proteins.

2. The method of claim 1, further comprising degrading nucleic acid.

3. The method of claim 2, wherein the one or more additional proteins inhibit degradation of at least a portion of the segment.

4. The method of claim 1, wherein the first and second proteins are the same.

5. The method of claim 1, wherein the first protein, the second protein, and the one or more additional proteins are Cas endonucleases.

6. The method of claim 5, wherein the Cas endonucleases are complexed with guide RNAs that are complementary to sequence at ends of a target nucleic acid.

7. The method of claim 6, wherein the Cas endonucleases are enzymatically inactive.

8. The method of claim 6, further comprising additional Cas endonuclease and associated guide RNA tiled in a region between said ends.

9. The method of claim 1, further comprising detecting at least a portion of the segment.

10. The method of claim 9, wherein the detecting step comprises DNA staining, spectrophotometry, sequencing, fluorescent probe hybridization, fluorescence resonance energy transfer, optical microscopy, or electron microscopy.

11. The method of claim 9, wherein the detecting step comprises detecting multiple portions of the segment.

12. The method of claim 11, further comprising:
obtaining sequencing reads from the multiple portions of the segment; and
mapping the sequence reads, thereby obtaining a sequence from a region of the segment that is longer than at least one of the portions of the segment.

13. The method of claim 1, wherein the ends of the segment are within one megabase.

14. The method of claim 1, wherein the nucleic acid sample is a blood sample, serum sample, plasma sample, urine sample, saliva sample, semen sample, feces sample, phlegm sample, or liquid biopsy.

15. The method of claim 14, wherein the sample is a plasma sample, and wherein the segment comprises cell-free DNA.

16. The method of claim 15, wherein the cell-free DNA comprises circulating tumor DNA.

17. The method of claim 14, wherein the nucleic acid sample is a plasma sample from a pregnant female, and wherein the segment comprises fetal DNA.

18. The method of claim 14, wherein the nucleic acid of interest comprises an insertion, deletion, substitution, inversion, amplification, duplication, translocation, or polymorphism.

19. The method of claim 14, wherein the nucleic acid sample is from an organism, and wherein the segment comprises a sequence foreign to a genome of the organism.

20. The method of claim 1, wherein the nucleic acid sample is selected from the group consisting of a soil sample, water sample, and food sample.
